(19) United States
(12) Patent Application Publication
(10) Pub. No.: US 2025/0352330 A1
(43) Pub. Date: Nov. 20, 2025

(54) REPLACEMENT HEART VALVE IMPLANT

(71) Applicant: BOSTON SCIENTIFIC SCIMED, INC., Maple Grove, MN (US)

(72) Inventors: Chenhao Fu, Plymouth, MN (US); Levi Joel Wolterstorff, Saint Paul, MN (US); Philip Andrew Litecky, Forest Lake, MN (US); Adam Troy Olson, Plymouth, MN (US)

(73) Assignee: BOSTON SCIENTIFIC SCIMED, INC., MAPLE GROVE, MN (US)

(21) Appl. No.: 19/207,839

(22) Filed: May 14, 2025

Related U.S. Application Data
(60) Provisional application No. 63/647,888, filed on May 15, 2024.

(51) Int. Cl. A61F 2/24

(52) U.S. Cl. CPC A61F 2/2418; A61F 2220/0016

(57) ABSTRACT

A replacement heart valve implant includes an expandable framework including a tubular wall comprising a plurality of interconnected struts and defining a central lumen, wherein the wall includes inflow and outflow ends, and a plurality of commissure posts. A plurality of valve leaflets is secured to the commissure posts. The framework may include a plurality of native leaflet clamps extending upstream from the outflow end, wherein the leaflet clamps are configured to pinch native leaflets of the native heart valve against the wall. The framework may include a plurality of native commissure clamps extending upstream from the commissure posts, wherein the commissure clamps are configured to pinch native commissures of the native heart valve. The framework may include a plurality of native leaflet clamps extending upstream from the commissure posts, wherein the leaflet clamps are configured to pinch native leaflets of the native heart valve against the wall.

### CROSS-REFERENCE TO RELATED APPLICATION

This application is a continuation of U.S. Patent Application Ser. No. 63/647,888, filed May 15, 2024, entitled “REPLACEMENT HEART VALVE IMPLANT”, which is incorporated by reference herein in its entirety.

### TECHNICAL FIELD

The disclosure relates generally to medical devices and more particularly to a replacement heart valve implant.

### BACKGROUND

A wide variety of intracorporeal medical devices have been developed for medical use including replacement heart valve implants for repair or replacement of diseased heart valves. Of the known medical devices and methods, each has certain advantages and disadvantages. There is an ongoing need to provide alternative medical devices as well as alternative methods for manufacturing and using medical devices.

### SUMMARY

In one example, a replacement heart valve implant for implantation within a native heart valve may comprise an expandable framework configured to shift between a delivery configuration and a deployed configuration, the expandable framework comprising a tubular wall comprising a plurality of interconnected struts and defining a central lumen extending along a central axis of the expandable framework, wherein the tubular wall comprises an inflow end and an outflow end, a plurality of commissure posts disposed circumferentially around the central axis, and a plurality of native leaflet clamps extending upstream from the outflow end of the tubular wall radially outward of the tubular wall, wherein the plurality of native leaflet clamps is configured to pinch native leaflets of the native heart valve against the tubular wall; and a plurality of valve leaflets disposed within the central lumen and secured to the plurality of commissure posts.

In addition, or alternatively, to any example disclosed herein, the plurality of native leaflet clamps comprises at least two native leaflet clamps disposed between a circumferentially adjacent pair of commissure posts of the plurality of commissure posts.

In addition, or alternatively, to any example disclosed herein, the plurality of native leaflet clamps comprises at least two native leaflet clamps disposed between each circumferentially adjacent pair of commissure posts of the plurality of commissure posts.

In addition, or alternatively, to any example disclosed herein, the plurality of native leaflet clamps is monolithically formed with the plurality of interconnected struts of the tubular wall.

In addition, or alternatively, to any example disclosed herein, in the deployed configuration, an upstream end of each native leaflet clamp of the plurality of leaflet clamps is disposed upstream of the outflow end of the tubular wall and downstream of the inflow end of the tubular wall.

In addition, or alternatively, to any example disclosed herein, the upstream end of each native leaflet clamp of the plurality of leaflet clamps is disposed radially outward of at least a portion of its respective native leaflet clamp.

In addition, or alternatively, to any example disclosed herein, the upstream end of each native leaflet clamp of the plurality of leaflet clamps is spaced apart radially outward from the tubular wall.

In addition, or alternatively, to any example disclosed herein, at least some leaflet clamps of the plurality of native leaflet clamps further comprise at least one barb extending therefrom, wherein the at least one barb is configured to engage with native valve leaflets of the native heart valve.

In addition, or alternatively, to any example disclosed herein, and in a second example, a replacement heart valve implant for implantation within a native heart valve may comprise an expandable framework configured to shift between a delivery configuration and a deployed configuration, the expandable framework comprising a tubular wall comprising a plurality of interconnected struts and defining a central lumen extending along a central axis of the expandable framework, wherein the tubular wall comprises an inflow end and an outflow end, a plurality of commissure posts disposed circumferentially around the central axis, and a plurality of native commissure clamps extending upstream from the plurality of commissure posts radially outward of the tubular wall, wherein the plurality of native commissure clamps is configured to pinch native commissures of the native heart valve; and a plurality of valve leaflets disposed within the central lumen and secured to the plurality of commissure posts.

In addition, or alternatively, to any example disclosed herein, the plurality of native commissure clamps comprises wires secured to and extending circumferentially between circumferentially adjacent pairs of commissure posts of the plurality of commissure posts.

In addition, or alternatively, to any example disclosed herein, each native commissure clamp of the plurality of native commissure clamps comprises a first wire and a second wire, wherein a first clamping portion of the first wire and a first clamping portion of the second wire are biased circumferentially toward each other.

In addition, or alternatively, to any example disclosed herein, the first wire further comprises a second clamping portion circumferentially spaced apart from the first clamping portion by a curved medial portion and the second wire further comprises a second clamping portion circumferentially spaced apart from the first clamping portion by a curved medial portion.

In addition, or alternatively, to any example disclosed herein, in the deployed configuration, the curved medial portion of the first wire and the curved medial portion of the second wire are disposed upstream of the outflow end of the tubular wall and downstream of the inflow end of the tubular wall.

In addition, or alternatively, to any example disclosed herein, in the deployed configuration, each wire further comprises at least one barb extending therefrom, wherein the at least one barb is configured to engage with native valve leaflets of the native heart valve.

In addition, or alternatively, to any example disclosed herein, each native commissure clamp of the plurality of native commissure clamps comprises a first clamping member extending upstream from one commissure post of the plurality of commissure posts to a first free end, and a second clamping member extending upstream from the one commissure post of the plurality of commissure posts to a second free end.

In addition, or alternatively, to any example disclosed herein, the first free end is self-biased toward the second free end to pinch one native valve commissure of the native heart valve between the first clamping member and the second clamping member.

In addition, or alternatively, to any example disclosed herein, the first free end comprises a first atraumatic tip and the second free end comprises a second atraumatic tip.

In addition, or alternatively, to any example disclosed herein, the first clamping member comprises at least one first barb extending toward the second clamping member, the at least one first barb being disposed between the one commissure post and the first free end.

In addition, or alternatively, to any example disclosed herein, the second clamping member comprises at least one second barb extending toward the first clamping member, the at least one second barb being disposed between the one commissure post and the second free end.

In addition, or alternatively, to any example disclosed herein, the plurality of native commissure clamps is monolithic with the expandable framework.

In addition, or alternatively, to any example disclosed herein, the plurality of native commissure clamps is formed separately from the expandable framework and configured to be attached to the expandable framework at a later time.

In addition, or alternatively, to any example disclosed herein, a replacement heart valve implant for implantation within a native heart valve may comprise an expandable framework configured to shift between a delivery configuration and a deployed configuration, the expandable framework comprising a tubular wall comprising a plurality of interconnected struts and defining a central lumen extending along a central axis of the expandable framework, wherein the tubular wall comprises an inflow end and an outflow end, a plurality of commissure posts disposed circumferentially around the central axis, and a plurality of native leaflet clamps extending upstream from the plurality of commissure posts radially outward of the tubular wall, wherein the plurality of native leaflet clamps is configured to pinch native leaflets of the native heart valve against the tubular wall; and a plurality of valve leaflets disposed within the central lumen and secured to the plurality of commissure posts.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects of a replacement heart valve implant according to the disclosure;

FIG. 3 illustrates selected aspects of a replacement heart valve implant according to the disclosure;

FIG. 4 illustrates selected aspects of a replacement heart valve implant according to the disclosure;

FIG. 5 illustrates selected aspects of a replacement heart valve implant according to the disclosure;

FIG. 6 illustrates selected aspects of a replacement heart valve implant according to the disclosure;

FIG. 7 illustrates selected aspects of a replacement heart valve implant according to the disclosure;

FIGS. 8-11 illustrates selected aspects of a portion of a replacement heart valve implant according to the disclosure; and

FIG. 12 illustrates selected aspects of a replacement heart valve implant according to the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

For the purpose of this disclosure, the discussion herein is directed toward use in treating a native heart valve such as the aortic valve and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

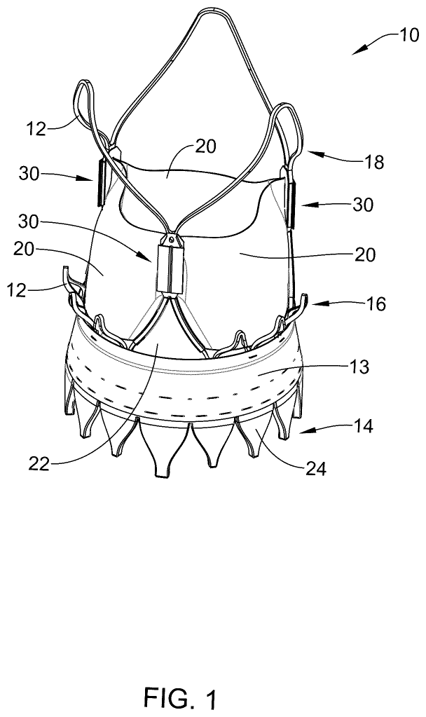

FIG. 1 illustrates selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 100 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) in a mammalian heart. The replacement heart valve implant 10 may be configured to allow one-way flow through the replacement heart valve implant 10 from an inflow end to an outflow end.

The replacement heart valve implant 10 may comprise an expandable framework 12. In some embodiments, the expandable framework 12 may have a substantially circular cross-section. In some embodiments, the expandable framework 12 may have a non-circular (e.g., D-shaped, elliptical, etc.) cross-section. In some embodiments, the expandable framework 12 may be formed from a resilient shape memory material. In at least some embodiments, the resilient shape memory material may be a metallic material such as nitinol. Some suitable but non-limiting examples of materials that may be used to form the expandable framework 12, including but not limited to metals and metal alloys, composites, polymers, and the like, are described below.

The replacement heart valve implant 10 and/or the expandable framework 12 may be configured to shift between a delivery configuration (e.g., a radially collapsed configuration) and a deployed configuration (e.g., a radially expanded configuration). In some embodiments, the expandable framework 12 may be self-expanding. In some embodiments, the expandable framework 12 may be self-biased toward the deployed configuration. In some embodiments, the expandable framework 12 may be mechanically expandable. In some embodiments, the expandable framework 12 may be balloon expandable. Other configurations are also contemplated. In some embodiments, the expandable framework 12 may include and/or define a plurality of interstices (e.g., openings) through the expandable framework 12.

In some embodiments, the expandable framework 12 may comprise a tubular wall 13 comprising a plurality of interconnected struts and defining a central lumen extending along a central axis of the expandable framework 12. In some embodiments, the tubular wall 13 may comprise and/or define an inflow end and an outflow end. In some embodiments, the expandable framework 12 and/or the tubular wall 13 may define a lower crown 14 proximate and/or at the inflow end, an upper crown 16 proximate and/or at the outflow end, and a plurality of stabilization arches 18 extending downstream from the outflow end. In some embodiments, the plurality of stabilization arches 18 may extend downstream of and/or away from the upper crown 16 in a direction opposite the lower crown 14. In some embodiments, the upper crown 16 may be disposed longitudinally and/or axially between the lower crown 14 and the plurality of stabilization arches 18.

In some embodiments, the expandable framework 12 may comprise a plurality of commissure posts 30. In at least some embodiments, the plurality of commissure posts 30 may be disposed downstream of the outflow end and/or the upper crown 16. In some embodiments, the plurality of commissure posts 30 may be disposed circumferentially around the central axis. In some embodiments, the plurality of commissure posts 30 may be disposed longitudinally and/or axially between the upper crown 16 and the plurality of stabilization arches 18. In some embodiments, the plurality of stabilization arches 18 may extend from the plurality of commissure posts 30.

In some embodiments, the replacement heart valve implant 10 may comprise a proximal portion and a distal portion. In some embodiments, orientation of the replacement heart valve implant 10 may be related to an implant delivery device and/or a direction of implantation relative to a target site (e.g., a native heart valve). In some embodiments, the proximal portion may comprise the outflow end and/or the plurality of stabilization arches 18. In some embodiments, the proximal portion may comprise the upper crown 16 and/or the plurality of commissure posts 30. In some embodiments, the distal portion may comprise the inflow end and/or the lower crown 14. Other configurations are also contemplated.

In some embodiments, the replacement heart valve implant 10 may comprise a plurality of valve leaflets 20 disposed within the central lumen. The plurality of valve leaflets 20 may be coupled, secured, and/or fixedly attached to the expandable framework 12 and/or the plurality of commissure posts 30. One or more means of securing the plurality of valve leaflets 20 to the expandable framework 12 and/or the plurality of commissure posts 30 may be used, including but not limited to, adhesive bonding, suturing, friction fit (e.g., pinching), etc. In some embodiments, the outflow end of the expandable framework 12 may comprise the plurality of stabilization arches 18 extending axially away from the plurality of valve leaflets 20 and/or from the plurality of commissure posts 30 or an attachment point (or attachment points) of the plurality of valve leaflets 20 with the expandable framework 12.

In some embodiments, each valve leaflet of the plurality of valve leaflets 20 may include a root edge coupled to the expandable framework 12 and a free edge (e.g., a coaptation edge) movable relative to the root edge to coapt with the free edges of the other leaflets along a coaptation region. In some embodiments, the plurality of valve leaflets 20 may be integrally formed with each other, such that the plurality of valve leaflets 20 is formed as a single unitary and/or monolithic unit. In some embodiments, the plurality of valve leaflets 20 may be formed integrally with other structures such as an inner skirt 22 and/or an outer skirt 24, base structures, liners, or the like.

The plurality of valve leaflets 20 may be configured to substantially restrict fluid from flowing through the replacement heart valve implant 10 and/or the central lumen in a closed position. For example, in some embodiments, the free edges of the plurality of valve leaflets 20 may move into coaptation with one another in the closed position to substantially restrict fluid from flowing through the replacement heart valve implant 10 and/or the central lumen. The free edges of the plurality of valve leaflets 20 may be moved apart from each other in an open position to permit fluid flow through the replacement heart valve implant 10 and/or the central lumen. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium, or other tissue. Other configurations and/or materials are also contemplated.

In some embodiments, the replacement heart valve implant 10 may include an inner skirt 22 disposed on and/or extending along an inner surface of the expandable framework 12 and/or the tubular wall 13. In at least some embodiments, the inner skirt 22 may be fixedly attached to the expandable framework 12 and/or the tubular wall 13. The inner skirt 22 may direct fluid, such as blood, flowing through the replacement heart valve implant 10 and/or the central lumen toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 may include an outer skirt 24 disposed on and/or extending along the outer surface of the expandable framework 12 and/or the tubular wall 13. In some embodiments, the outer skirt 24 may be disposed at and/or adjacent the lower crown 14. The outer skirt 24 may ensure the fluid flows through the central lumen of the replacement heart valve implant 10 and does not flow around the replacement heart valve implant 10 (e.g., between the expandable framework 12 and the vessel wall), so as to ensure that the plurality of valve leaflets 20 can stop the flow of fluid when in the closed position.

In some embodiments, the inner skirt 22 and/or the outer skirt 24 may include a polymer, and/or may include at least 50 percent by weight of a polymer. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of the plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 23 millimeters (mm) (0.906 inches (in)), about 25 mm (0.984 in), about 27 mm (1.063 in), about 30 mm (1.181 in), etc. in an unconstrained configuration (e.g., in the deployed configuration). In some embodiments, the expandable framework 12 and/or the replacement heart valve implant 10 may have an outer extent of about 10 mm (0.394 in), about 9 mm (0.354 in) about 8 mm (0.315 in), about 7 mm (0.276 in), about 6 mm (0.236 in), etc. in the delivery configuration. Other configurations are also contemplated.

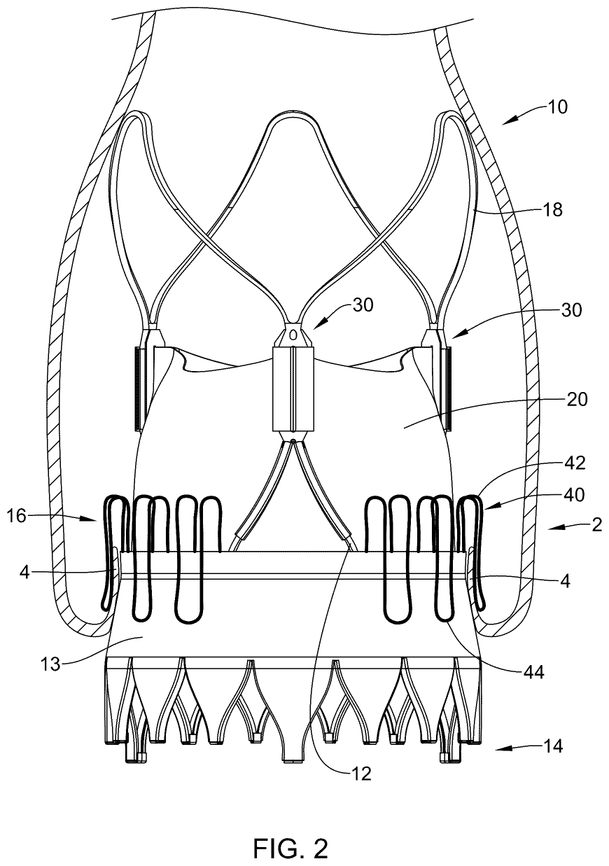

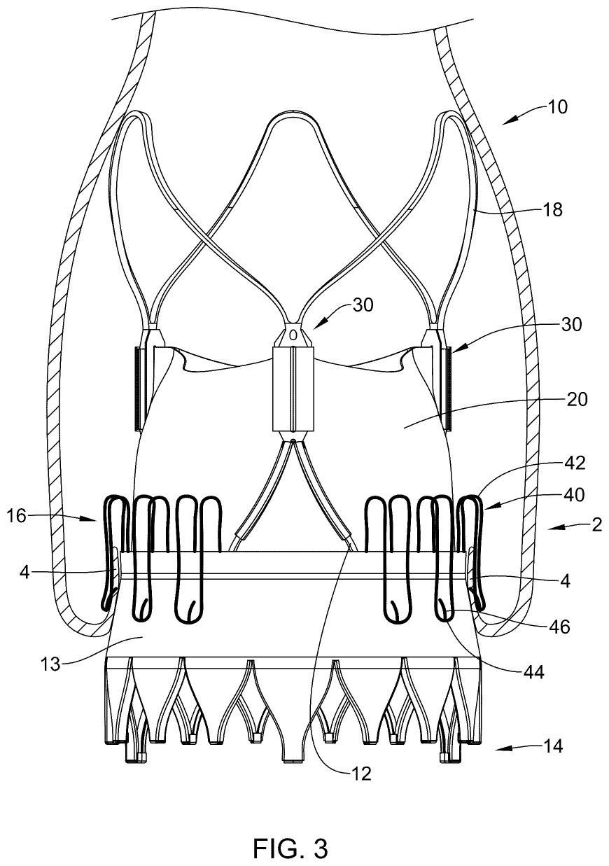

FIGS. 2-3 illustrates selected aspects of some embodiments of the replacement heart valve implant 10 in accordance with the current disclosure. Some features and/or elements that are not expressly shown and/or discussed with respect to FIGS. 2-3 may be shown in FIG. 1 and/or discussed above. Some features and/or elements may be hidden from view. In the interest of clarity, the replacement heart valve implant 10 is shown in a partial cutaway side view, where the replacement heart valve implant 10 is disposed within a native heart valve 2 (e.g., a native aortic valve, etc.) comprising a plurality of native leaflets 4.

In some embodiments, the expandable framework 12 may comprise a plurality of native leaflet clamps 40 extending upstream from the outflow end of the tubular wall 13 radially outward of the tubular wall 13 in the deployed configuration. In at least some embodiments, the plurality of native leaflet clamps 40 may be configured to pinch native leaflets 4 of the native heart valve 2 against an outer surface of the tubular wall 13 in the deployed configuration.

In some embodiments, the plurality of native leaflet clamps 40 may be monolithically formed with the expandable framework 12 and/or the plurality of interconnected struts of the tubular wall 13. In some embodiments, the plurality of native leaflet clamps 40 may be formed in place of and/or as extensions of the upper crown 16. In at least some embodiments, in the deployed configuration, at least a portion of each native leaflet clamp of the plurality of native leaflet clamps 40 may extend downstream of and/or from the tubular wall 13 before doubling back at a downstream bend 42 (e.g., a proximal bend) to extend in an upstream direction (e.g., a distal direction) toward and/or to an upstream end 44 (e.g., a distal end).

In some embodiments, the downstream bend 42 may bend each native leaflet clamp of the plurality of native leaflet clamps 40 about 180 degrees. In some embodiments, the downstream bend 42 may bend each native leaflet clamp of the plurality of native leaflet clamps 40 more than 180 degrees such that an upstream extending medial portion of each native leaflet clamp of the plurality of native leaflet clamps 40 extends radially inward toward the tubular wall 13. In some alternative embodiments, the upstream extending medial portion of each native leaflet clamp of the plurality of native leaflet clamps 40 may extend generally parallel to the central axis of the expandable framework 12 and/or generally parallel to the tubular wall 13. Other configurations are also contemplated.

In some embodiments, in the deployed configuration, the upstream end 44 of each native leaflet clamp of the plurality of native leaflet clamps 40 may be disposed upstream of the outflow end of the tubular wall 13 and downstream of the inflow end of the tubular wall 13. In some embodiments, in the deployed configuration, the upstream end 44 of each native leaflet clamp of the plurality of native leaflet clamps 40 may be disposed radially outward of at least a portion of its respective native leaflet clamp. For example, the upstream end 44 and/or an upstream end portion extending from the upstream extending medial portion to the upstream end 44 may be bent radially outward and/or may be angled away from the central axis of the expandable framework 12 and/or the tubular wall 13 in the upstream direction (e.g., in the distal direction). In some embodiments, in the deployed configuration, the upstream end 44 of each native leaflet clamp of the plurality of native leaflet clamps 40 (e.g., a distalmost tip of each native leaflet clamp of the plurality of native leaflet clamps 40) may be spaced apart radially outward from the tubular wall 13.

In some embodiments, the plurality of native leaflet clamps 40 may comprise at least two native leaflet clamps disposed between a circumferentially adjacent pair of commissure posts of the plurality of commissure posts 30. In some embodiments, the plurality of native leaflet clamps 40 may comprise at least two native leaflet clamps disposed between each circumferentially adjacent pair of commissure posts of the plurality of commissure posts 30. In some embodiments, the at least two native leaflet clamps may comprise exactly two native leaflet clamps, three native leaflet clamps, four native leaflet clamps, etc. disposed between a circumferentially adjacent pair of commissure posts of the plurality of commissure posts 30 and/or disposed between each circumferentially adjacent pair of commissure posts of the plurality of commissure posts 30. Other configurations are also contemplated.

In some embodiments, the at least two native leaflet clamps disposed between a circumferentially adjacent pair of commissure posts of the plurality of commissure posts 30 may constitute a grouping of native leaflet clamps, wherein the plurality of native leaflet clamps 40 may comprise a plurality of groupings of native leaflet clamps spaced circumferentially around the central axis of the expandable framework 12. In at least some embodiments, the plurality of groupings of native leaflet clamps may be equally spaced circumferentially around the central axis of the expandable framework 12. Other configurations are also contemplated. In some embodiments, each grouping of native leaflet clamps may comprise an equal number of native leaflet clamps. In some embodiments, at least one grouping of native leaflet clamps may comprise a different number of native leaflet clamps than another grouping, or other groupings, of native leaflet clamps. Other configurations are also contemplated.

In some embodiments, at least one native leaflet clamp of the plurality of native leaflet clamps 40 may further comprise at least one barb 46 extending therefrom, as seen in FIG. 3. In some embodiments, at least some native leaflet clamps of the plurality of native leaflet clamps 40 may further comprise at least one barb 46 extending therefrom. In some embodiments, each native leaflet clamp of the plurality of native leaflet clamps 40 may further comprise at least one barb 46 extending therefrom. In some embodiments, the at least one barb 46 may comprise exactly one barb, two barbs, three barbs, four barbs, etc.

In some embodiments, the at least one barb 46 may be configured to engage with native leaflets 4 of the native heart valve 2. In some embodiments, the at least one barb 46 may be configured to penetrate native leaflets 4 of the native heart valve 2. Other configurations are also contemplated.

In some embodiments, the at least one barb 46 (or a subset thereof) may extend radially inward from the upstream end 44 of its respective native leaflet clamp. In some embodiments, the at least one barb 46 (or a subset thereof) may extend downstream, and/or toward the outflow end, from the upstream end 44 of its respective native leaflet clamp. In some embodiments, the at least one barb 46 (or a subset thereof) may extend upstream, and/or toward the inflow end, from the upstream end 44 of its respective native leaflet clamp. Other configurations, including combinations thereof, are also contemplated.

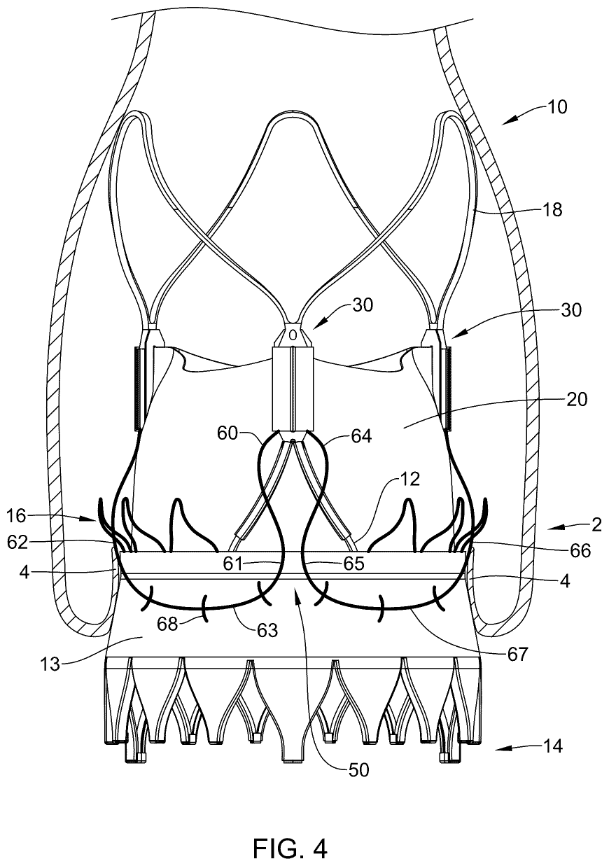

FIG. 4 illustrates selected aspects of an embodiment of the replacement heart valve implant 10 in accordance with the current disclosure. Some features and/or elements that are not expressly shown and/or discussed with respect to FIG. 4 may be shown in FIG. 1 and/or discussed above. Some features and/or elements may be hidden from view. In the interest of clarity, the replacement heart valve implant 10 is shown in a partial cutaway side view, where the replacement heart valve implant 10 is disposed within a native heart valve 2 (e.g., a native aortic valve, etc.) comprising a plurality of native leaflets 4.

In some embodiments, the expandable framework 12 may comprise a plurality of native commissure clamps 50 extending upstream from the plurality of commissure posts 30 radially outward of the tubular wall 13 in the deployed configuration. In at least some embodiments, the plurality of native commissure clamps 50 may be configured to pinch native commissures of the native heart valve 2, thereby preventing downstream movement of the replacement heart valve implant 10 with respect to the native heart valve 2. In some embodiments, the plurality of native commissure clamps 50 may form and/or may serve as native commissure saddles configured to span and/or straddle the native commissures of the native heart valve 2, thereby preventing upstream movement of the replacement heart valve implant 10 with respect to the native heart valve 2. In some embodiments, the plurality of native commissure clamps 50 may also be configured to pinch native leaflets 4 of the native heart valve 2 against an outer surface of the tubular wall 13 in the deployed configuration.

In some embodiments, the plurality of native commissure clamps 50 may be monolithic with the expandable framework 12 and/or the plurality of commissure posts 30. In some alternative embodiments, the plurality of native commissure clamps 50 may be formed separately from the expandable framework 12 and/or the plurality of commissure posts 30, and the plurality of native commissure clamps 50 may be configured to be attached and/or secured to the expandable framework 12 and/or the plurality of commissure posts 30 at a later time. Other configurations are also contemplated.

In some embodiments, the plurality of native commissure clamps 50 may comprise wires secured to and extending circumferentially between circumferentially adjacent pairs of commissure posts of the plurality of commissure posts 30. In some embodiments, the plurality of native commissure clamps 50 may comprise wires secured to and extending from one commissure post (e.g., a first commissure post) to a circumferentially adjacent commissure post (e.g., a second commissure post) of the plurality of commissure posts 30.

In some embodiments, each native commissure clamp of the plurality of native commissure clamps 50 may be fixedly attached to and/or monolithically formed with a mounting plate (not shown) configured to be attached to and/or secured to the plurality of commissure posts 30. In some embodiments, the mounting plate(s) may be configured to be attached and/or secured to the plurality of commissure posts 30 radially outward of the plurality of commissure posts 30. Other configurations are also contemplated.

In some embodiments, each commissure clamp of the plurality of native commissure clamps 50 may comprise a first wire 60 (extending to the left of its respective commissure post when viewed looking toward the central axis from outside of the tubular wall 13) and a second wire 64 (extending to the right of its respective commissure post when viewed looking toward the central axis from outside of the tubular wall 13). In some embodiments, the first wire 60 may comprise a first clamping portion 61 and a second clamping portion 62 circumferentially spaced apart from the first clamping portion 61 by a curved medial portion 63. Similarly, the second wire 64 may comprise a first clamping portion 65 and a second clamping portion 66 circumferentially spaced apart from the first clamping portion 65 by a curved medial portion 67 extending circumferentially around the tubular wall 13.

In at least some embodiments, the first clamping portion 61 of the first wire 60 and the first clamping portion 65 of the second wire 64 may be biased circumferentially toward each other in the deployed configuration. In at least some embodiments, the first clamping portion 61 of the first wire 60 and the first clamping portion 65 of the second wire 64 may be self-biased circumferentially toward each other in the deployed configuration. Accordingly, the first clamping portion 61 of the first wire 60 and the first clamping portion 65 of the second wire 64 may be configured to pinch a native valve commissure therebetween in the deployed configuration.

In some embodiments, in the deployed configuration, the curved medial portion 63 of the first wire 60 and the curved medial portion 67 of the second wire 64 are disposed upstream of the outflow end of the tubular wall 13 and downstream of the inflow end of the tubular wall 13. In some embodiments, the curved medial portion 63 of the first wire 60 may form an arcuate shape that is generally concave in the downstream direction and/or in a radially inward direction (e.g., toward the central axis). In some embodiments, the curved medial portion 67 of the second wire 64 may each form an arcuate shape that is generally concave in the downstream direction and/or in a radially inward direction (e.g., toward the central axis). Other configurations are also contemplated.

In some embodiments, in the deployed configuration, each wire (e.g., the first wire 60, the second wire 64, etc.) may optionally comprise at least one barb 68 extending therefrom. In some embodiments, in the deployed configuration, each curved medial portion (e.g., the curved medial portion 63 of the first wire 60, the curved medial portion 67 of the second wire 64, etc.) may optionally comprise at least one barb 68 extending therefrom. In some embodiments, in the deployed configuration, the at least one barb 68 may extend radially inward toward the central axis and/or the tubular wall 13. In some embodiments, in the deployed configuration, the at least one barb 68 may extend upstream and/or toward the inflow end from its respective wire (e.g., the first wire 60, the second wire 64, etc.). In some embodiments, in the deployed configuration, the at least one barb 68 may extend downstream and/or toward the outflow end from its respective wire (e.g., the first wire 60, the second wire 64, etc.). In some embodiments, in the deployed configuration, the at least one barb 68 may extend upstream and downstream from its respective wire (e.g., the first wire 60, the second wire 64, etc.). In some embodiments, the at least one barb 68 may be configured to engage with native leaflets 4 of the native heart valve 2. It shall be understood that the at least one barb 68 is not required in all embodiments and the wires (e.g., the first wire 60, the second wire 64, etc.) may be devoid of the at least one barb 68 and/or any other structure projecting therefrom.

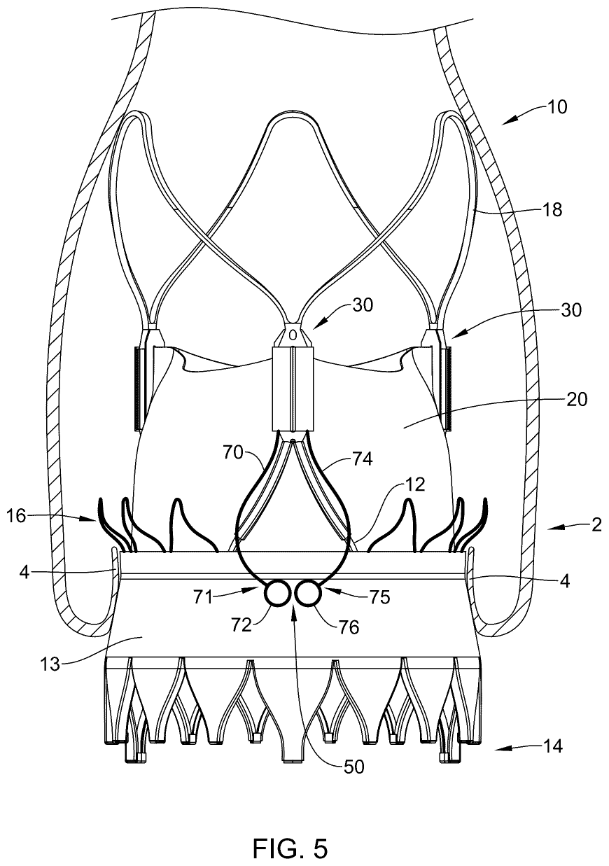

FIGS. 5-11 illustrates selected aspects of embodiments of the replacement heart valve implant 10 in accordance with the current disclosure. Some features and/or elements that are not expressly shown and/or discussed with respect to FIGS. 5-11 may be shown in FIG. 1 and/or discussed above. Some features and/or elements may be hidden from view. In the interest of clarity, the replacement heart valve implant 10 is shown in a partial cutaway side view, where the replacement heart valve implant 10 is disposed within a native heart valve 2 (e.g., a native aortic valve, etc.) comprising a plurality of native leaflets 4.

In some embodiments, the expandable framework 12 may comprise a plurality of native commissure clamps 50 extending upstream from the plurality of commissure posts 30 radially outward of the tubular wall 13 in the deployed configuration. To improve clarity in the figures, only one native commissure clamp is illustrated, but it shall be understood that additional commissure clamps may be present in any embodiment. In some embodiments, the plurality of native commissure clamps 50 may comprise exactly two native commissure clamps, three native commissure clamps, etc. In at least some embodiments, the plurality of native commissure clamps 50 may be configured to pinch native commissures of the native heart valve 2, thereby preventing downstream movement of the replacement heart valve implant 10 with respect to the native heart valve 2. In some embodiments, the plurality of native commissure clamps 50 may form and/or may serve as native commissure saddles configured to span and/or straddle the native commissures of the native heart valve 2, thereby preventing upstream movement of the replacement heart valve implant 10 with respect to the native heart valve 2.

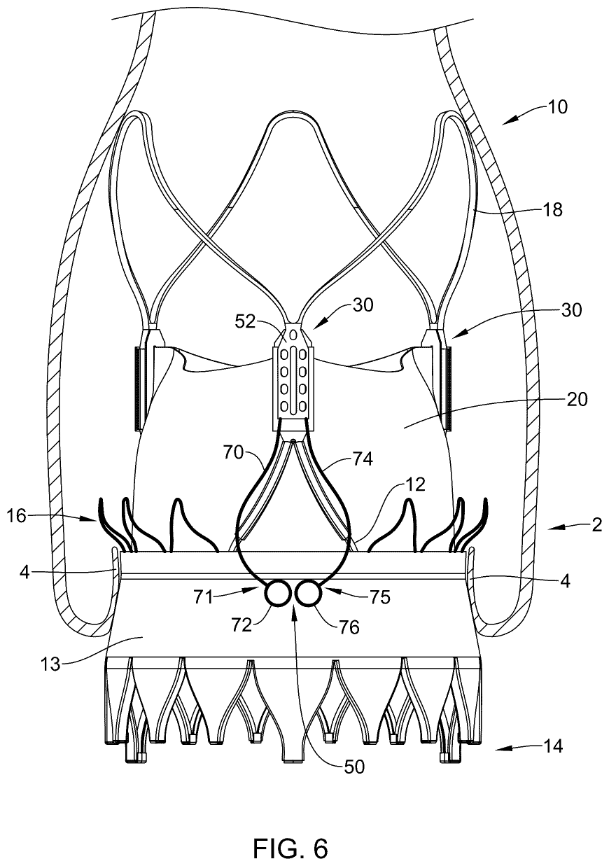

In some embodiments, the plurality of native commissure clamps 50 may be monolithic with the expandable framework 12 and/or the plurality of commissure posts 30, as seen in FIG. 5. In some embodiments, the plurality of native commissure clamps 50 may be formed separately from the expandable framework 12 and/or the plurality of commissure posts 30, and the plurality of native commissure clamps 50 may be configured to be attached and/or secured to the expandable framework 12 and/or the plurality of commissure posts 30 at a later time. In some embodiments, the plurality of native commissure clamps 50 may each be fixedly attached to and/or monolithically formed with a mounting plate 52 configured to be attached to and/or secured to the plurality of commissure posts 30, as seen in FIGS. 6-11. In some embodiments, the mounting plate 52 may be configured to be attached and/or secured to the plurality of commissure posts 30 radially outward of the plurality of commissure posts 30. In some embodiments, the mounting plate 52 may be configured to be attached and/or secured to the plurality of commissure posts 30 using adhesive bonding, suturing, snap-on and/or clip-on features, or other suitable means. Other configurations are also contemplated.

In some embodiments, each commissure clamp of the plurality of native commissure clamps 50 may comprise a first clamping member 70 (extending to the left of its respective commissure post when viewed looking toward the central axis from outside of the tubular wall 13) extending upstream from one commissure post of the plurality of commissure posts 30 to a first free end 71 radially outward of the tubular wall 13 in the deployed configuration, and a second clamping member 74 (extending to the right of its respective commissure post when viewed looking toward the central axis from outside of the tubular wall 13) extending upstream from the one commissure post of the plurality of commissure posts 30 to a second free end 75 radially outward of the tubular wall 13 in the deployed configuration. In some embodiments, the first clamping member 70 may comprise a first wire and the second clamping member 74 may comprise a second wire. Other configurations are also contemplated.

In at least some embodiments, the first clamping member 70 and/or the first free end 71 may be biased circumferentially toward the second clamping member 74 and/or the second free end 75 in the deployed configuration. In at least some embodiments, the first clamping member 70 and/or the first free end 71 may be self-biased circumferentially toward the second clamping member 74 and/or the second free end 75 in the deployed configuration. Accordingly, the first clamping member 70 and/or the first free end 71 and the second clamping member 74 and/or the second free end 75 may be configured to pinch one native valve commissure of the native heart valve 2 between the first clamping member 70 and the second clamping member 74, and/or between the first free end 71 and the second free end 75, in the deployed configuration.

In some embodiments, the first clamping member 70 and/or the first free end 71 may comprise a first atraumatic tip 72 configured to avoid and/or prevent penetration into tissue and/or the native valve commissure. In some embodiments, the second clamping member 74 and/or the second free end 75 may comprise a second atraumatic tip 76 configured to avoid and/or prevent penetration into tissue and/or the native valve commissure. In some embodiments, the first atraumatic tip 72 and/or the second atraumatic tip 76 may be formed and/or configured as a closed loop, as seen in FIGS. 5-6. Other configurations are also contemplated.

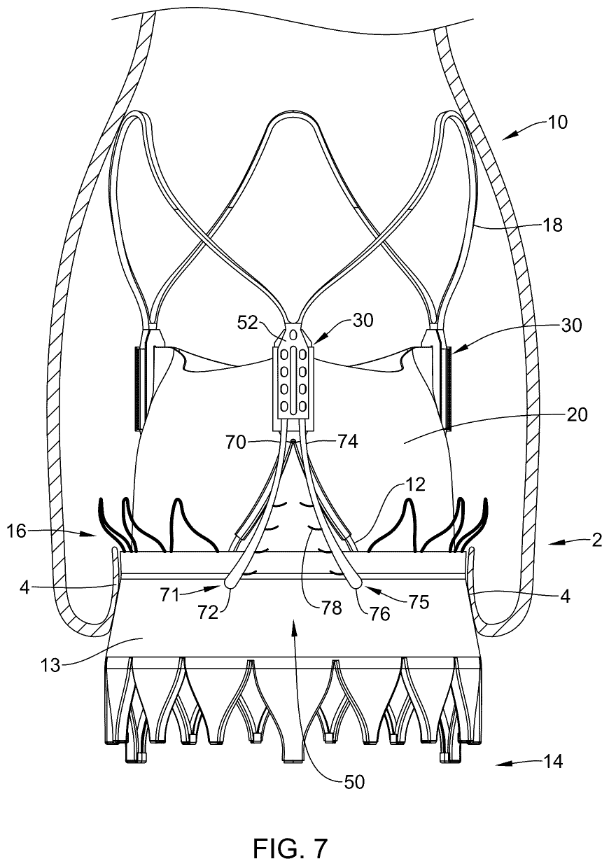

In some embodiments, in the deployed configuration, the first clamping member 70 may optionally comprise at least one barb 78 extending circumferentially from the first clamping member 70 toward the second clamping member 74, as seen in FIG. 7. In some embodiments, the at least one barb 78 of the first clamping member 70 may be disposed between the one commissure post of the plurality of commissure posts 30 and the first free end 71.

In some embodiments, in the deployed configuration, the second clamping member 74 may optionally comprise at least one barb 78 extending circumferentially from the second clamping member 74 toward the first clamping member 70, as seen in FIG. 7. In some embodiments, the at least one barb 78 of the second clamping member 74 may be disposed between the one commissure post of the plurality of commissure posts 30 and the second free end 75.

In some embodiments, in the deployed configuration, the at least one barb 78 may extend radially inward toward the central axis and/or the tubular wall 13. In some embodiments, in the deployed configuration, the at least one barb 78 may extend upstream and/or toward the inflow end. In some embodiments, in the deployed configuration, the at least one barb 78 may extend downstream and/or toward the outflow end. In some embodiments, in the deployed configuration, the at least one barb 78 may extend upstream and downstream. In some embodiments, the at least one barb 78 may be configured to engage with a native valve commissure of the native heart valve 2. It shall be understood that the at least one barb 78 is not required in all embodiments and the first clamping member 70 and/or the second clamping member 74 may be devoid of the at least one barb 78 and/or any other structure projecting therefrom.

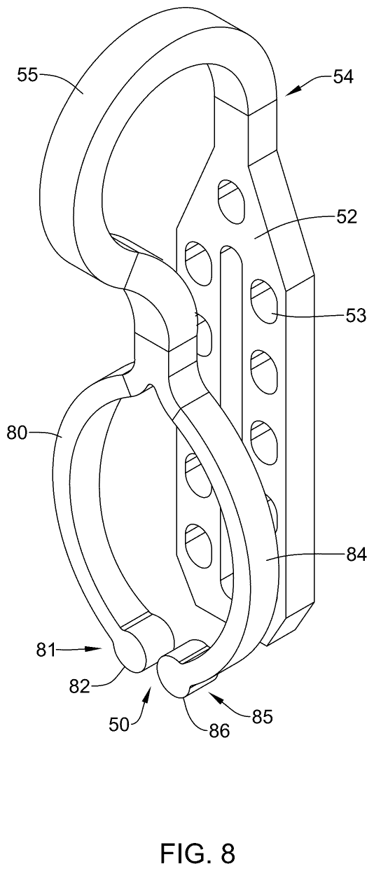

FIGS. 8-11 illustrate selected aspects of additional embodiments of singular examples of the plurality of native commissure clamps 50 that are similar in function to the plurality of native commissure clamps 50 of FIG. 6. In some embodiments, each commissure clamp of the plurality of native commissure clamps 50 may be fixedly attached to and/or monolithically formed with a mounting plate 52 configured to be attached to and/or secured to the plurality of commissure posts 30. In some embodiments, the mounting plate 52 may be formed with and/or may comprise a plurality of apertures 53 formed therein. In some embodiments, the mounting plate 52 may be configured to be attached and/or secured to the plurality of commissure posts 30 radially outward of the plurality of commissure posts 30. In some embodiments, the mounting plate 52 may be configured to be attached and/or secured to the plurality of commissure posts 30 using adhesive bonding, suturing, snap-on and/or clip-on features, or other suitable means. In some alternative embodiments, the mounting plate 52 may be configured to be attached and/or secured directly to the expandable framework 12 and/or the plurality of stabilization arches 18. In some embodiments, the mounting plate 52 may be configured to be attached and/or secured directly to the expandable framework 12 and/or the plurality of stabilization arches 18 using adhesive bonding, suturing, snap-on and/or clip-on features, or other suitable means. Other configurations are also contemplated.

In some embodiments, each commissure clamp of the plurality of native commissure clamps 50 may comprise an arm 54 fixedly attached to and/or monolithically formed with the mounting plate 52. In some embodiments, each commissure clamp of the plurality of native commissure clamps 50 may comprise a first clamping member 80 and a second clamping member 84. In some embodiments, the arm 54 may extend from the mounting plate 52 to the first clamping member 80 and the second clamping member 84.

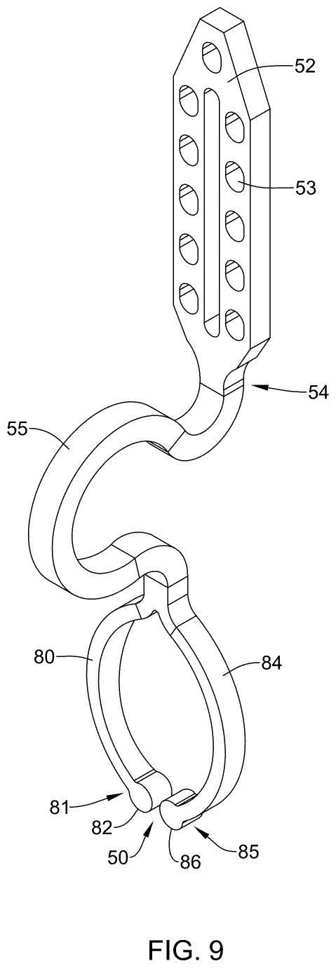

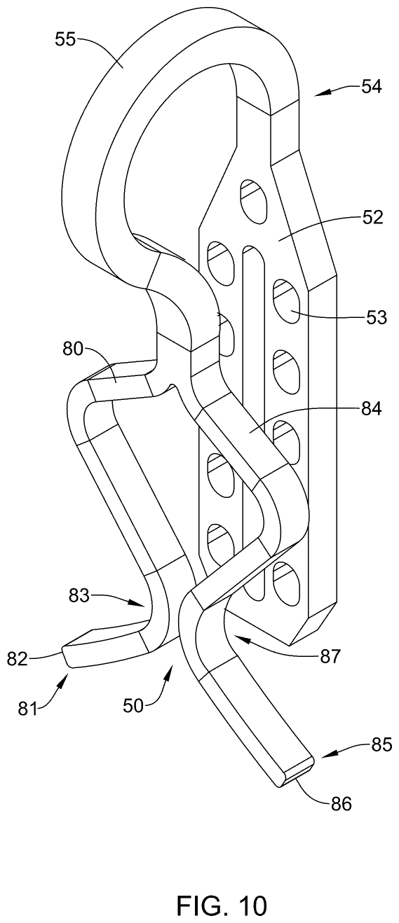

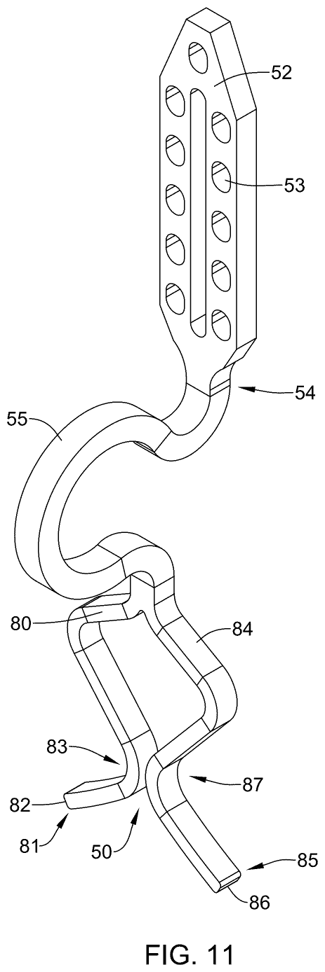

In some embodiments, the arm 54 may extend downstream from the mounting plate 52 (e.g., in the downstream direction), as seen in FIGS. 8 and 10, to a looping portion 55 that doubles back to the upstream direction to the first clamping member 80 and the second clamping member 84. In some embodiments, the arm 54 may extend upstream from the mounting plate 52 (e.g., in the upstream direction), as seen in FIGS. 9 and 11, to a looping portion 55 that extends in the upstream direction to the first clamping member 80 and the second clamping member 84. In some embodiments, the looping portion 55 may permit the native commissure clamp(s) to flex to accommodate anatomical differences and/or to accommodate variation in implant depth relative to the annulus of the native heart valve 2.

In some embodiments, the first clamping member 80 may extend from the arm 54 upstream toward and/or to a first free end 81 radially outward of the tubular wall 13 in the deployed configuration, and the second clamping member 84 may extend from the arm 54 upstream toward and/or to a second free end 85 radially outward of the tubular wall 13 in the deployed configuration.

In at least some embodiments, the first clamping member 80 and/or the first free end 81 may be biased circumferentially toward the second clamping member 84 and/or the second free end 85 in the deployed configuration. In at least some embodiments, the first clamping member 80 and/or the first free end 81 may be self-biased circumferentially toward the second clamping member 84 and/or the second free end 85 in the deployed configuration. Accordingly, the first clamping member 80 and/or the first free end 81 and the second clamping member 84 and/or the second free end 85 may be configured to pinch one native valve commissure of the native heart valve 2 between the first clamping member 80 and the second clamping member 84, and/or between the first free end 81 and the second free end 85, in the deployed configuration.

In some embodiments, the first clamping member 80 and/or the first free end 81 may comprise a first atraumatic tip 82 configured to avoid and/or prevent penetration into tissue and/or the native valve commissure. In some embodiments, the second clamping member 84 and/or the second free end 85 may comprise a second atraumatic tip 86 configured to avoid and/or prevent penetration into tissue and/or the native valve commissure. Other configurations are also contemplated.

In some embodiments, the first clamping member 80 and/or the second clamping member 84 may be arcuate in a circumferential direction, as seen in FIGS. 8 and 9. In some embodiments, the first clamping member 80 may be devoid of bends and/or other structure between the arm 54 and the first free end 81. In some embodiments, the second clamping member 84 may be devoid of bends and/or other structure between the arm 54 and the second free end 85.

In some embodiments, the first clamping member 80 and/or the second clamping member 84 may have a zig-zag shape and/or an S-shape in a circumferential direction, as seen in FIGS. 10 and 11. In some embodiments, the first clamping member 80 may comprise one or more bends and/or other structure between the arm 54 and the first free end 81. In some embodiments, the second clamping member 84 may comprise one or more bends and/or other structure between the arm 54 and the second free end 85.

In some embodiments, the first free end 81 may extend circumferentially away from the second free end 85. In some embodiments, a first bend 83 disposed between the arm 54 and/or the looping portion 55 and the first free end 81 may be biased circumferentially toward a second bend 87 disposed between the arm 54 and/or the looping portion 55 and the second free end 85. In some embodiments, the first bend 83 disposed between the arm 54 and/or the looping portion 55 and the first free end 81 may be self-biased circumferentially toward the second bend 87 disposed between the arm 54 and/or the looping portion 55 and the second free end 85. Accordingly, the first clamping member 80 and/or the first bend 83 and the second clamping member 84 and/or the second bend 87 may be configured to pinch one native valve commissure of the native heart valve 2 between the first clamping member 80 and the second clamping member 84, and/or between the first bend 83 and the second bend 87, in the deployed configuration.

In some embodiments, in the deployed configuration, the first clamping member 80 may optionally comprise at least one barb (not shown) extending circumferentially from the first clamping member 80 toward the second clamping member 84. In some embodiments, the at least one barb of the first clamping member 80 may be disposed between the arm 54 and/or the looping portion 55 and the first free end 81.

In some embodiments, in the deployed configuration, the second clamping member 84 may optionally comprise at least one barb (not shown) extending circumferentially from the second clamping member 84 toward the first clamping member 80. In some embodiments, the at least one barb of the second clamping member 84 may be disposed between the arm 54 and/or the looping portion 55 and the second free end 85.

In some embodiments, in the deployed configuration, the at least one barb may extend radially inward toward the central axis and/or the tubular wall 13. In some embodiments, in the deployed configuration, the at least one barb may extend upstream and/or toward the inflow end. In some embodiments, in the deployed configuration, the at least one barb may extend downstream and/or toward the outflow end. In some embodiments, in the deployed configuration, the at least one barb may extend upstream and downstream. In some embodiments, the at least one barb may be configured to engage with a native valve commissure of the native heart valve 2. It shall be understood that the at least one barb is not required in all embodiments and the first clamping member 80 and/or the second clamping member 84 may be devoid of the at least one barb and/or any other structure projecting therefrom.

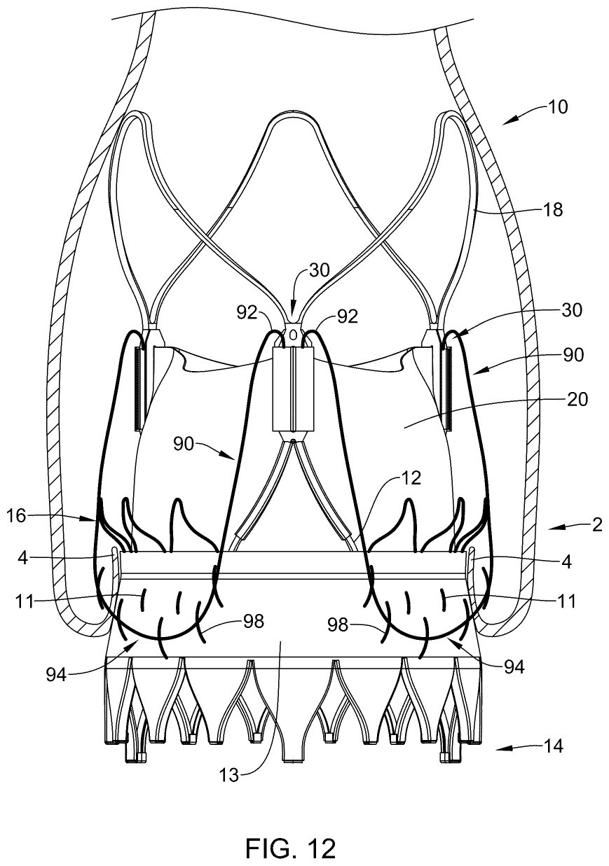

FIG. 12 illustrates selected aspects of an embodiment of the replacement heart valve implant 10 in accordance with the current disclosure. Some features and/or elements that are not expressly shown and/or discussed with respect to FIG. 12 may be shown in FIG. 1 and/or discussed above. Some features and/or elements may be hidden from view. In the interest of clarity, the replacement heart valve implant 10 is shown in a partial cutaway side view, where the replacement heart valve implant 10 is disposed within a native heart valve 2 (e.g., a native aortic valve, etc.) comprising a plurality of native leaflets 4.

In some embodiments, the expandable framework 12 may comprise a plurality of native leaflet clamps 90 extending upstream from the outflow end of the tubular wall 13 radially outward of the tubular wall 13 in the deployed configuration. In some embodiments, the expandable framework 12 may comprise a plurality of native leaflet clamps 90 extending upstream from the plurality of commissure posts 30 radially outward of the tubular wall 13 in the deployed configuration. In at least some embodiments, the plurality of native leaflet clamps 90 may be configured to pinch native leaflets 4 of the native heart valve 2 against an outer surface of the tubular wall 13 in the deployed configuration.

In some embodiments, the plurality of native leaflet clamps 90 may be monolithically formed with the expandable framework 12 and/or the plurality of commissure posts 30. In some alternative embodiments, the plurality of native leaflet clamps 90 may be formed separately from the expandable framework 12 and/or the plurality of commissure posts 30, and the plurality of native leaflet clamps 90 may be configured to be attached and/or secured to the expandable framework 12 and/or the plurality of commissure posts 30 at a later time. Other configurations are also contemplated.

In some embodiments, the plurality of native leaflet clamps 90 may comprise wires secured to and extending circumferentially between circumferentially adjacent pairs of commissure posts of the plurality of commissure posts 30. In some embodiments, the plurality of native leaflet clamps 90 may comprise wires secured to and extending from one commissure post (e.g., a first commissure post) to a circumferentially adjacent commissure post (e.g., a second commissure post) of the plurality of commissure posts 30.

In some embodiments, each native leaflet clamp of the plurality of native leaflet clamps 90 may be fixedly attached to and/or monolithically formed with a mounting plate (not shown) configured to be attached to and/or secured to the plurality of commissure posts 30. In some embodiments, the mounting plate(s) may be configured to be attached and/or secured to the plurality of commissure posts 30 radially outward of the plurality of commissure posts 30. Other configurations are also contemplated.

In at least some embodiments, in the deployed configuration, at least a portion of each native leaflet clamp of the plurality of native leaflet clamps 90 may extend downstream of and/or from at attachment point to the plurality of commissure posts 30 and/or the mounting plate associated with its respective native leaflet clamp before doubling back at a downstream bend 92 (e.g., a proximal bend) to extend in an upstream direction (e.g., a distal direction) toward and/or to an upstream end 94 (e.g., a distal end).

In some embodiments, the downstream bend 92 may bend each native leaflet clamp of the plurality of native leaflet clamps 90 about 180 degrees. In some embodiments, the downstream bend 92 may bend each native leaflet clamp of the plurality of native leaflet clamps 90 more than 180 degrees (e.g., between about 180 degrees and about 210 degrees) such that an upstream extending medial portion of each native leaflet clamp of the plurality of native leaflet clamps 90 extends radially inward toward the tubular wall 13. In some embodiments, the downstream bend 92 may bend each native leaflet clamp of the plurality of native leaflet clamps 90 less than 180 degrees (e.g., between about 150 degrees and about 180 degrees) such that an upstream extending medial portion of each native leaflet clamp of the plurality of native leaflet clamps 90 extends radially outward away from the tubular wall 13. In some embodiments, the upstream extending medial portion of each native leaflet clamp of the plurality of native leaflet clamps 90 may extend generally parallel to the central axis of the expandable framework 12 and/or generally parallel to the tubular wall 13. Other configurations are also contemplated.

In some embodiments, in the deployed configuration, the upstream end 94 of each native leaflet clamp of the plurality of native leaflet clamps 90 may be disposed upstream of the outflow end of the tubular wall 13 and downstream of the inflow end of the tubular wall 13. In some embodiments, in the deployed configuration, the upstream end 94 of each native leaflet clamp of the plurality of native leaflet clamps 90 may be disposed radially outward of at least a portion of its respective native leaflet clamp. For example, the upstream end 94 and/or an upstream end portion extending from the upstream extending medial portion to the upstream end 94 may be bent radially outward and/or may be angled away from the central axis of the expandable framework 12 and/or the tubular wall 13 in the upstream direction (e.g., in the distal direction). In some embodiments, in the deployed configuration, the upstream end 94 of each native leaflet clamp of the plurality of native leaflet clamps 40 (e.g., a distalmost tip of each native leaflet clamp of the plurality of native leaflet clamps 40) may be spaced apart radially outward from the tubular wall 13.

In some embodiments, the plurality of native leaflet clamps 90 may comprise at least one native leaflet clamp disposed between a circumferentially adjacent pair of commissure posts of the plurality of commissure posts 30. In some embodiments, the plurality of native leaflet clamps 90 may comprise at least one native leaflet clamp disposed between each circumferentially adjacent pair of commissure posts of the plurality of commissure posts 30. Other configurations are also contemplated.

In some embodiments, in the deployed configuration, at least one native leaflet clamp of the plurality of native leaflet clamps 90 may optionally comprise at least one barb 98 extending therefrom, as seen in FIG. 12. In some embodiments, in the deployed configuration, at least some native leaflet clamps of the plurality of native leaflet clamps 90 may optionally comprise at least one barb 98 extending therefrom. In some embodiments, in the deployed configuration, each native leaflet clamp of the plurality of native leaflet clamps 90 may optionally comprise at least one barb 98 extending therefrom. In some embodiments, the at least one barb 98 may comprise exactly one barb, two barbs, three barbs, four barbs, etc.

In some embodiments, in the deployed configuration, the at least one barb 98 (or a subset thereof) may extend radially inward from the upstream end 94 of its respective native leaflet clamp. In some embodiments, in the deployed configuration, the at least one barb 98 (or a subset thereof) may extend downstream, and/or toward the outflow end, from the upstream end 94 of its respective native leaflet clamp. In some embodiments, in the deployed configuration, the at least one barb 98 (or a subset thereof) may extend upstream, and/or toward the inflow end, from the upstream end 94 of its respective native leaflet clamp. Other configurations, including combinations thereof, are also contemplated.

In some embodiments, in the deployed configuration, the replacement heart valve implant 10 may comprise at least one leaflet barb 11 extending radially outward from the tubular wall 13. In some embodiments, in the deployed configuration, the at least one leaflet barb 11 may extend downstream and/or toward the outflow end. In some embodiments, in the deployed configuration, the at least one leaflet barb 11 may extend upstream and/or toward the inflow end. Other configurations, including combinations thereof, are also contemplated.

In some embodiments, the at least one leaflet barb 11 and/or the at least one barb 98 may be configured to engage with native leaflets 4 of the native heart valve 2. In some embodiments, at least one leaflet barb 11 and/or the at least one barb 98 may be configured to penetrate native leaflets 4 of the native heart valve 2. Other configurations are also contemplated. It shall be understood that at least one leaflet barb 11 and/or the at least one barb 98 is not required in all embodiments. For example, in some embodiments, the plurality of native leaflet clamps 90 may be devoid of the at least one barb 98 and/or any other structure projecting therefrom, and/or the tubular wall 13 may be devoid of the at least one leaflet barb 11.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten and alloys thereof, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A replacement heart valve implant for implantation within a native heart valve, comprising:
an expandable framework configured to shift between a delivery configuration and a deployed configuration, the expandable framework comprising:
a tubular wall comprising a plurality of interconnected struts and defining a central lumen extending along a central axis of the expandable framework, wherein the tubular wall comprises an inflow end and an outflow end;
a plurality of commissure posts disposed circumferentially around the central axis; and
a plurality of native leaflet clamps extending upstream from the outflow end of the tubular wall radially outward of the tubular wall, wherein the plurality of native leaflet clamps is configured to pinch native leaflets of the native heart valve against the tubular wall;
and
a plurality of valve leaflets disposed within the central lumen and secured to the plurality of commissure posts.

2. The replacement heart valve implant of claim 1, wherein the plurality of native leaflet clamps comprises at least two native leaflet clamps disposed between a circumferentially adjacent pair of commissure posts of the plurality of commissure posts.

3. The replacement heart valve implant of claim 2, wherein the plurality of native leaflet clamps comprises at least two native leaflet clamps disposed between each circumferentially adjacent pair of commissure posts of the plurality of commissure posts.

4. The replacement heart valve implant of claim 1, wherein the plurality of native leaflet clamps is monolithically formed with the plurality of interconnected struts of the tubular wall.

5. The replacement heart valve implant of claim 1, wherein in the deployed configuration, an upstream end of each native leaflet clamp of the plurality of leaflet clamps is disposed upstream of the outflow end of the tubular wall and downstream of the inflow end of the tubular wall.

6. The replacement heart valve implant of claim 5, wherein the upstream end of each native leaflet clamp of the plurality of leaflet clamps is disposed radially outward of at least a portion of its respective native leaflet clamp.

7. The replacement heart valve implant of claim 5, wherein the upstream end of each native leaflet clamp of the plurality of leaflet clamps is spaced apart radially outward from the tubular wall.

8. The replacement heart valve implant of claim 1, wherein at least some leaflet clamps of the plurality of native leaflet clamps further comprise at least one barb extending therefrom, wherein the at least one barb is configured to engage with native valve leaflets of the native heart valve.

9. A replacement heart valve implant for implantation within a native heart valve, comprising:
an expandable framework configured to shift between a delivery configuration and a deployed configuration, the expandable framework comprising:
a tubular wall comprising a plurality of interconnected struts and defining a central lumen extending along a central axis of the expandable framework, wherein the tubular wall comprises an inflow end and an outflow end;
a plurality of commissure posts disposed circumferentially around the central axis; and
a plurality of native commissure clamps extending upstream from the plurality of commissure posts radially outward of the tubular wall, wherein the plurality of native commissure clamps is configured to pinch native commissures of the native heart valve;
and
a plurality of valve leaflets disposed within the central lumen and secured to the plurality of commissure posts.

10. The replacement heart valve implant of claim 9, wherein the plurality of native commissure clamps comprises wires secured to and extending circumferentially between circumferentially adjacent pairs of commissure posts of the plurality of commissure posts.

11. The replacement heart valve implant of claim 10, wherein each native commissure clamp of the plurality of native commissure clamps comprises a first wire and a second wire, wherein a first clamping portion of the first wire and a first clamping portion of the second wire are biased circumferentially toward each other.

12. The replacement heart valve implant of claim 11, wherein the first wire further comprises a second clamping portion circumferentially spaced apart from the first clamping portion by a curved medial portion and the second wire further comprises a second clamping portion circumferentially spaced apart from the first clamping portion by a curved medial portion.

13. The replacement heart valve implant of claim 12, wherein in the deployed configuration, the curved medial portion of the first wire and the curved medial portion of the second wire are disposed upstream of the outflow end of the tubular wall and downstream of the inflow end of the tubular wall.

14. The replacement heart valve implant of claim 10, wherein in the deployed configuration, each wire further comprises at least one barb extending therefrom, wherein the at least one barb is configured to engage with native valve leaflets of the native heart valve.

15. The replacement heart valve implant of claim 9, wherein each native commissure clamp of the plurality of native commissure clamps comprises a first clamping member extending upstream from one commissure post of the plurality of commissure posts to a first free end, and a second clamping member extending upstream from the one commissure post of the plurality of commissure posts to a second free end;
wherein the first free end is self-biased toward the second free end to pinch one native valve commissure of the native heart valve between the first clamping member and the second clamping member.

16. The replacement heart valve implant of claim 15, wherein the first free end comprises a first atraumatic tip and the second free end comprises a second atraumatic tip.

17. The replacement heart valve implant of claim 15, wherein the first clamping member comprises at least one first barb extending toward the second clamping member, the at least one first barb being disposed between the one commissure post and the first free end; and
wherein the second clamping member comprises at least one second barb extending toward the first clamping member, the at least one second barb being disposed between the one commissure post and the second free end.

18. The replacement heart valve implant of claim 9, wherein the plurality of native commissure clamps is monolithic with the expandable framework.

19. The replacement heart valve implant of claim 9, wherein the plurality of native commissure clamps is formed separately from the expandable framework and configured to be attached to the expandable framework at a later time.

20. A replacement heart valve implant for implantation within a native heart valve, comprising:
an expandable framework configured to shift between a delivery configuration and a deployed configuration, the expandable framework comprising:
a tubular wall comprising a plurality of interconnected struts and defining a central lumen extending along a central axis of the expandable framework, wherein the tubular wall comprises an inflow end and an outflow end;
a plurality of commissure posts disposed circumferentially around the central axis; and
a plurality of native leaflet clamps extending upstream from the plurality of commissure posts radially outward of the tubular wall, wherein the plurality of native leaflet clamps is configured to pinch native leaflets of the native heart valve against the tubular wall;
and
a plurality of valve leaflets disposed within the central lumen and secured to the plurality of commissure posts.
